(19) United States
(12) Patent Application Publication
(10) Pub. No.: US 2011/0092453 A1
(43) Pub. Date: Apr. 21, 2011

(54) TREATMENTS AND PREVENTION OF HYDROCEPHALUS

(75) Inventors: Jaleel Ahmed Miyan, Manchester (GB); Penelope Jane Owen-Lynch, Lancaster (GB)

(73) Assignee: THE UNIVERSITY OF MANCHESTER, Manchester (GB)

(21) Appl. No.: 12/672,111

(22) PCT Filed: Aug. 7, 2008
(86) PCT No.: PCT/GB08/02680

§ 371 Date: Jul. 28, 2010

(30) Foreign Application Priority Data
Aug. 8, 2007 (GB) 0715502.1

(51) Int. Cl. A61K 31/505; A61K 31/495; A61P 25/00

(52) U.S. Cl. 514/50; 514/249

(57) ABSTRACT

The present invention relates to the prevention or treatment of hydrocephalus. In one embodiment, the invention includes the use of one or more bioavailable folate derivatives, or salts thereof, for the prevention or treatment of hydrocephalus. Examples of more bioavailable folate derivatives that may be used include any combination of: folinic acid, tetrahydrofolate, thymidine, 10-formyltetrahydrofolate or methyltetrahydrofolate, or salts thereof. The invention also relates to a composition comprising two or more bioavailable folate derivative(s); for example, folinic acid and tetrahydrofolate.

The present invention relates to methods for preventing and treating hydrocephalus.

Hydrocephalus (HC) is a condition with multifactor aetiology resulting from an imbalance in the production and/or absorption of cerebrospinal fluid (CSF) within the head and spinal column. This leads to an accumulation of fluid within the ventricles and fluid spaces within and around the brain.

The most devastating form of HC, early-onset HC (EOHC), is that seen in foetuses and new born infants which, according to the National Institutes of Health statistics for the USA, affects 1 in 1000 live human births, making this a very significant neurological problem. Similar rates have been reported for other western countries. However rates of up to 1 in 100 have been reported in some parts of the third world (e.g. in Asia, Arabia, Persia, Africa).

Because hydrocephalus injures the brain, thought and behavior may be adversely affected. Learning disabilities are common among those with hydrocephalus, who tend to score better on verbal IQ than on performance IQ, which is thought to reflect the distribution of nerve damage to the brain. However, the severity of hydrocephalus differs considerably between individuals and some are of average or above average intelligence. Someone with hydrocephalus may have motivation and visual problems, problems with co-ordination, and may be clumsy. They may hit puberty earlier than the average. About one in four patients develop epilepsy.

Symptoms of hydrocephalus vary with age, disease progression, and individual differences in tolerance to CSF. For example, an infant's ability to tolerate CSF pressure differs from an adult's. The infant skull can expand to accommodate the build up of CSF because the sutures (the fibrous joints that connect the bones of the skull) have not yet closed. In infancy, the most obvious indication of hydrocephalus is often the rapid increase in head circumference or an unusually large head size. Other symptoms may include vomiting, sleepiness, irritability, downward deviation of the eyes (also called “sunsetting”), and seizures. Older children and adults may experience different symptoms because their skulls cannot expand to accommodate the build up of CSF.

In older children or adults, symptoms may include headache followed by vomiting, nausea, papilledema (swelling of the optic disk which is part of the optic nerve), blurred vision, diplopia (double vision), sunsetting of the eyes, problems with balance, poor coordination, gait disturbance, urinary incontinence, slowing or loss of development, lethargy, drowsiness, irritability, or other changes in personality or cognition including memory loss.

Hydrocephalus may be diagnosed before birth by prenatal ultrasound, a diagnostic imaging technique which uses high-frequency sound waves and a computer to create, images of blood vessels, tissues, and organs. Ultrasounds are used to view internal organs as they function, and to assess blood flow through various vessels. In many cases, hydrocephalus does not develop until the third trimester of the pregnancy and, therefore, may not be seen on ultrasounds performed earlier in pregnancy. Also, atypical cases are not detected until other clinical symptoms become obvious. Symptoms vary with age, disease progression, and individual differences in tolerance to CSF accumulation. Furthermore, ultrasound diagnosis often results in false positives, as enlarged ventricles can occur as part of normal development and then return to normal size. Such false positives can lead to unnecessary terminations.

Hydrocephalus may also be diagnosed through clinical neurological evaluation and by using cranial imaging techniques, including computer tomography scanning (CT scans), which may be used frequently to evaluate the condition of the disorder throughout the patient's life. Magnetic resonance imaging (MRI) imaging can also be used. It is important to note that each CT scan exposes the patient to many times the level of x-ray radiation than that of a chest x-ray. Further commonly used tests include transillumination of the head, which can show abnormal fluid accumulation; lumbar puncture and examination of the CSF; skull X-ray; brain scan using radioisotopes which can show abnormalities in the fluid pathway; an arteriography of brain blood vessels.

Existing treatment methods for hydrocephalus usually involve surgical drainage of CSF through insertion of ventriculo-peritoneal shunts or ventriculostomy. It is estimated that about 35,000-45,000 shunt surgeries are performed each year in the USA, and that about 250,000 patients currently live with hydrocephalus. Despite shunting many of these patients have major neurological deficits which are linked to abnormal development of the cerebral cortex during the foetal stages of life. Furthermore, shunting can lead to complications: shunt malfunction, shunt failure, and shunt infection. Although a shunt generally works well, it may stop working if it disconnects, becomes blocked, or it is outgrown. If this happens the cerebrospinal fluid will begin to accumulate again and a number of physical symptoms will develop; some extremely serious, like seizures. The shunt failure rate is also relatively high and it is not uncommon for patients to have multiple shunt revisions within their lifetime. The diagnosis of cerebrospinal fluid buildup is complex and requires expertise. It is also important to note that there is no cure for hydrocephalus; it is a life-long disorder that can severely affect the quality of life.

As mentioned above, existing treatments for hydrocephalus are limited to surgical intervention. In view of the disadvantages inherent in surgical treatment, it is clear that there is a need to identify agents that will prevent the development of, and treat, hydrocephalus.

An aspect of the present invention provides one or more bioavailable folate derivatives, or salts thereof, for the prevention or treatment of hydrocephalus.

A further aspect of the present invention provides the use of one or more bioavailable folate derivatives, or salts thereof, in the manufacture of a medicament for the prevention or treatment of hydrocephalus.

A further aspect of the invention provides a method of preventing or treating hydrocephalus comprising administering one or more bioavailable folate derivatives, or salts thereof, to a subject in need of said treatment.

By “hydrocephalus” we include any hydrocephalus occurring in the fetal or neonatal peroid including, but not limited to, Early Onset hydrocephalus (EOHC), Fetal Onset hydrocephalus, Congenital hydrocephalus, Obstructive hydrocephalus, Communicating hydrocephalus. Preferably the hydrocephalus is Early Onset hydrocephalus.

By “preventing or treating” we include where the invention can both prevent the occurrence of hydrocephalus, as well as alleviating or reversing neurological defects arising from hydrocephalus. Thus, a subject having hydrocephalus, when treated according to the invention, may be able to enjoy a better quality of life by having more normal brain development.

By “subject” we include any animal that is susceptible to hydrocephalus, preferably a vertebrate, more preferably a mammal such as a domesticated farmyard animal or a human being. Most preferably the subject is a human being. As discussed below, the subject may be a pregnant female, a developing fetus, a neonate, an infant, a child, an adolescent or an adult.

The inventors have conducted extensive studies into the causes of hydrocephalus, in particular early onset hydrocephalus (EOHC), using a hydrocephalic Texas rat (H-Tx) model of this condition. H-Tx is a widely recognised, well-characterised model of hydrocephalus that mirrors features of human hydrocephalus (Table 1).

The inventors identified that abnormal development of the cortex in H-Tx rats occurs following obstruction of CSF flow. Abnormal CSF flow is linked to a lack of proliferation of cortical germinal epithelium (GE) cells, resulting in reduced output of neurones into the cortical plate. This lack of GE cell proliferation is caused by inhibition of the GE cells replication, as removing cells from the hydrocephalic brain and placing them into standard growth medium “releases them” from their in vivo arrested state and they show a normal ability to divide.

Moreover, incubating normal GE cells with CSF from hydrocephalic rat fetuses causes inhibition of normal GE cell proliferation. Importantly, CSF from human hydrocephalic infants also inhibits proliferation of rat cortical neurons growing in culture. These studies suggest that components of human and rat CSF are similar in nature and effect.

Therefore the inventors have identified that one or more components of CSF from hydrocephalic rats and humans directly affects germinal epithelium (GE) stem cell replication, thus reducing output of neurones into the cortex. The general reduction in cell division within the brain and fluid spaces also leads directly to hydrocephalus. One possible mechanism is through a reduction in the generation of output (drainage) channels for CSF. Hence, changes in CSF composition cause hydrocephalus.

By examining the protein composition of cerebral spinal fluid (CSF) of normal and affected rats, to their surprise the inventors found that the enzyme formyltetrahydrofolate dehydrogenase (FMTHFDH) is present in the CSF of hydrocephalic rats.

FMTHFDH acts to remove excess folate from the body (e.g. in the blood or liver) and as such plays an important role in regulating folate metabolism. In hydrocephalic rats FMTHFDH accumulates in the CSF, thereby likely reducing the amount of bioavailable folate derivatives available to cells. Although not wishing to be bound by any particular theory, the inventors consider that the reduction in GE cell proliferation in the cortex is due to alterations in the metabolism of folates within the CSF. Therefore the inventors conclude reduced levels of bioavailable folate derivatives in the CSF cause the poor brain development seen in hydrocephalus and furthermore may be causative in the induction of CSF accumulation.

Folate (or folic acid) is an essential source of key metabolites for the biosynthesis of amino acids and nucleic acids. It is a very important dietary component during periods of rapid cell division and growth, such as infancy and pregnancy. Adequate folic acid intake during the periconceptional period, the time just before and just after a woman becomes pregnant, helps protect against a number of congenital malformations, including neural tube defects, e.g spina bifida and anencephaly. The discovery of a link between insufficient folic acid and neural tube defects (NTDs) has led in some countries to food fortification, in which folic acid is added to foodstuffs with the intention of everyone benefiting from the associated rise in blood folate levels. Since the introduction of folic acid fortified foods, the rate of neural tube defects dropped by 25 percent in the United States. However, it is important to point out that hydrocephalus is not a neural tube defect. Hydrocephalus has a different physical manifestation and a different cause.

The link between bioavailable folate derivates and hydrocephalus is surprising as the folate fortification program in the US did not reduce hydrocephalus instance in the population. Therefore the skilled person, looking for a cause of hydrocephalus, would not have considered folate metabolism to be linked to hydrocephalus.

The inventors hypothesise that the reason why folic acid does not have an effect on preventing hydrocephalus is due to an imbalance of metabolism of folates within the hydrocephalic CSF caused by the presence of FMTHFDH. This surprising finding would not have been apparent prior to the inventors demonstrating that excess FMTHFDH is present in hydrocephalic CSF. This surprising finding also explains why the folic acid fortification program mentioned above did not reduce hydrocephalus in incidence in the US population.

Since FMTHFDH alters folate metabolism, the inventors reasoned that it would be beneficial to prevent or treat hydrocephalus by supplying a subject with one or more bioavailable folate derivates; preferably, the derivative is not a substrate for the FMTHFDH. As shown in the accompanying examples, the inventors have successfully demonstrated using the H-Tx rat model that bioavailable folate derivatives can reduce the instance of hydrocephalus. Thus bioavailable folate derivates can be used successfully to prevent hydrocephalus, at least in rat. It is important to point out that this is the first successful demonstration of a preventative treatment for hydrocephalus.

By “bioavailable folate derivative” we include any derivative of folate that can be used directly, or metabolised within the CSF to generate a derivative that can be taken up directly, into a cell.

The term folate can be used in two different ways. Folate, a member of the B-vitamin family, can be used as a collective term for a number of chemical forms, which are structurally related and which have similar biological activity to folic acid. Folate is also the term that can be used for the anionic form of folic acid. Folic acid is a synthetic folate form which is used for food fortification and nutritional supplements. It is not one of the principal naturally occurring forms of folate, used in the collective sense.

In the present application, by “folate” we mean the anionic form of folic acid. Folate/folic acid is therefore not a derivative of folate. Hence the term “bioavailable folate derivative” does not include folate/folic acid. For the reasons out lined above, folic acid does not have an effect on preventing hydrocephalus.

Examples of bioavailable folate derivates suitable for use in the invention include folinic acid, tetrahydrofolate, thymidine, 10-formyltetrahydrofolate and methyltetrahydrofolate, or salts and combinations thereof. Preferably the bioavailable folate derivative is folinic acid, or a non-toxic salt of folinic acid, for example the calcium or sodium folinate salts of folinic acid.

The invention also includes combinations of one or more bioavailable folate derivatives, or salts thereof. For example, as discussed further below, a combination of folinic acid with tetrahydrofolate (which can be in the form of tetrahydrofolic acid) may be particularly effective for the prevention or treatment of hydrocephalus. While not wishing to be bound by any particular theory, the inventors consider that such a combination is beneficial for the synthesis of both nucleic acids and proteins in a cell. Thus an embodiment of the invention is wherein the bioavailable folate derivative(s) comprises a combination of folinic acid and tetrahydrofolate (tetrahydrofolic acid).

Folinic Acid

Folinic acid is a 5-formyl-derivative of tetrahydrofolic acid. It is important to point out that folinic acid would not have been considered for the prevention or treatment of hydrocephalus until the inventors surprising finding of a link between bioavailable folate derivates and hydrocephalus.

Folinic acid has the systematic (IUPAC) name of 2-[4-[(2-amino-5-formyl-4-oxo-5,6,7,8-tetrahydro-1H-pteridin-6-yl)methylaminobenzoyl]aminopentanedioic acid, a formula of C₂₀H₂₃N₇O₇ and a molecular weight of 473.44 g/mol. The formula for folinic acid is presented below:

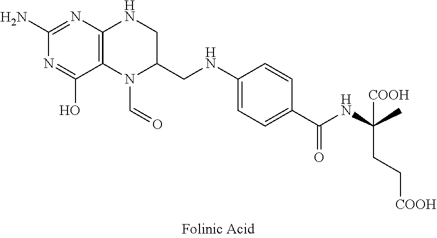

Folinic acid is known as an adjuvant in cancer chemotherapy involving the drug methotrexate, and is generally administered as calcium folinate or sodium folinate (commonly known as leucovorin). It is also used in synergistic combination with the chemotherapy 5-fluorouracil. There has been no reported uses of folinic acid in the prevention or treatment of hydrocephalus.

Folinic acid can be obtained from many sources, including Sigma (folinic acid calcium salt; catalogue number F8259).

Folinic acid, or a salt thereof, can be used in the present invention to both treat hydrocephalus and as a prophylactic to prevent hydrocephalus. In the following passages, unless stated otherwise a reference to “folinic acid” also encompasses the salts of folinic acid set out above.

Tetrahydrofolate

Tetrahydrofolate is the main active metabolite of dietary folate. It is vital as a coenzyme in reactions involving transfers of single carbon groups. Examples of pathways in which tetrahydrofolate has a role include: purine synthesis; pyrimidine synthesis; amino acid conversions: histidine to glutamic acid, homocysteine to methionine, serine to glycine. As nucleic and amino acid synthesis is affected by a deficiency of tetrahydrofolate, actively dividing and growing cells tend to be the first affected. Tetrahydrofolate can be in the acid form, tetrahydrofolic acid, and any reference herein to tetrahydrofolate includes tetrahydrofolic acid.

Tetrahydrofolate is fully called 5,6,7,8-Tetrahydrofolate, formula of C₁₉H₂₃N₇O₆ and a molecular weight of 445.43 g/mol. The formula for tetrahydrofolate is presented below:

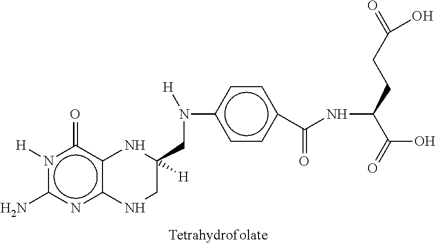

Tetrahydrofolate can be used in the present invention to both treat hydrocephalus and as a prophylactic to prevent hydrocephalus. In the following passages, unless stated otherwise a reference to “tetrahydrofolate” also encompasses any salts of this compound.

Tetrahydrofolate can be obtained from many different sources, including Sigma (tetrahydrofolic acid; catalogue number T3125).

Thymidine

Thymidine (deoxythymidine) is a pyrimidine deoxynucleoside compound. It is formed of a deoxyribose (a pentose sugar) joined to the pyrimidine base thymine. Deoxythymidine is non-toxic and as part of one of the four nucleotides in DNA it is a naturally occurring compound that exists in all living organisms.

Thymidine has the formula C₁₀H₁₄N₂O₅ and has a molecular weight of 242.23 g/mol. The formula for thymidine is given below.

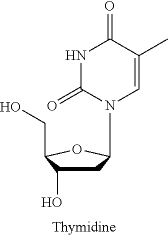

Thymidine can be obtained from many sources, including Sigma (catalogue number 1895).

Thymidine can be used in the present invention to both treat hydrocephalus and as a prophylactic to prevent hydrocephalus. In the following passages, unless stated otherwise a reference to “thymidine” also encompasses any salts of this compound.

10-formyltetrahydrofolate

10-formyltetrahydrofolate has the formula C₂₀H₂₁N₇O₇ and has a molecular weight of 473.17 g/mol. formula for 10-formyltetrahydrofolate is given below.

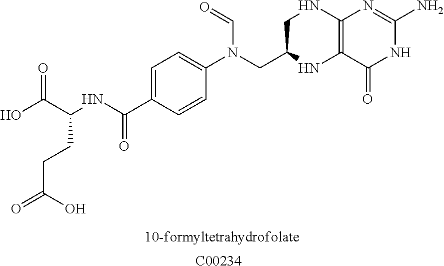

10-formyltetrahydrofolate can be used in the present invention to both treat hydrocephalus and as a prophylactic to prevent hydrocephalus. In the following passages, unless stated otherwise a reference to “10-formyltetrahydrofolate” also encompasses any salts of this compound.

10-formyltetrahydrofolate can be readily synthesised using well known laboratory methods: see, for example, Rabinowitz, J. C. (1963) Methods Enzymol. 6, 814-816, as described in: Krupenko, S. A., Wagner, C., and Cook, R. J. (1997) J. Biol. Chem. 272, 10266-1027.

Methyltetrahydrofolate

Methyltetrahydrofolate (MTHF) is the predominant form of folate in cerebrospinal fluid. Measuring MTHF levels in the CSF is useful to determine a deficiency of folate in central nervous system tissue. Low CSF MTHF levels are associated with inborn errors of metabolism affecting folate metabolism and in dietary deficiency of folate. Methyltetrahydrofolate is involved in a number of biosynthetic pathways, including the synthesis of the amino acid methionine. Methyltetrahydrofolate can be in the acid form, 5-methyl tetrahydrofolic acid, and any reference herein to methyltetrahydrofolate includes 5-methyl tetrahydrofolic acid.

Methyltetrahydrofolate (5-methyl-5,6,7,8-tetrahydrofolate) has the formula C₂₀H₂₅N₇O₆ and a molecular weight of 459.461 g/mol. The formula for methyltetrahydrofolate is presented below:

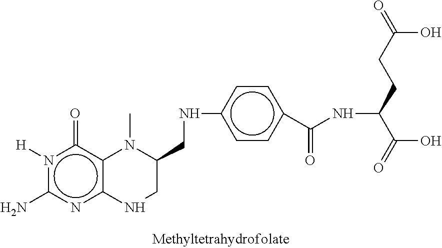

Methyltetrahydrofolate can be used in the present invention to both treat hydrocephalus and as a prophylactic to prevent hydrocephalus. In the following passages, unless stated otherwise a reference to “methyltetrahydrofolate” also encompasses any salts of this compound.

Methyltetrahydrofolate can be obtained from many different sources, including Sigma (5-methyl tetrahydrofolic acid; catalogue number M0132).

As mentioned above, combinations of one or more of the bioavailable folate derivates provided herein can be used in the aspects of the invention. Such combinations are not known or suggested in the prior art.

For example, as shown in the accompanying examples in vitro toxicity studies suggest that a combination of folinic acid and tetrahydrofolate is not toxic to cells. Further data seems to show that that combination can also reverse the development of hydrocephalus and improved cortex development. While not wishing to be bound by any particular theory, the inventors consider that such a combination is beneficial since the compounds can be used directly by cells as part of nucleic acid and protein synthesis without the need for elaborate metabolic conversion. Therefore a combination of folinic acid and tetrahydrofolate has a surprising benefit not known or suggested from the prior art.

Methods of diagnosing hydrocephalus are set out above in previous paragraphs of this specification. Using such information the skilled person would readily be able to diagnose whether a subject is suffering from hydrocephalus. By “subject” we include a pregnant female, a developing fetus, a neonate, an infant, a child, an adolescent or an adult.

As mentioned above, one or more bioavailable folate derivates can be used to both prevent and treat hydrocephalus.

There are several ways in which the bioavailable folate derivates can be used as a preventative measure for hydrocephalus.

One use of the bioavailable folate derivates is wherein the derivative(s) are administered to a subject planning to be pregnant, possibly in the form of a dietary supplement. Such a supplement could be formulated as tablets or capsules, in a similar way to presently available pre-pregnancy dietary supplements. Also, bioavailable folate derivates can be used to fortify foodstuffs to ensure that all consumers benefit from an increase in dietary bioavailable folate derivates, including pregnant women. Hence an embodiment of the invention is wherein the bioavailable folate derivate(s), or medicament comprising the bioavailable folate derivative(s), is formulated as a dietary supplement.

Food fortification with folic acid was recently introduced in several countries, including the United States. In 1996, the US Food and Drug Administration (FDA) published regulations requiring the addition of folic acid to enriched breads, cereals, flours, corn meals, pastas, rice, and other grain products. As cereals and grains are widely consumed in the U.S., these products have become a very important contributor of folic acid to the American diet. Since the addition of folic acid in grain-based foods as mandated by the Food and Drug Administration, the rate of neural tube defects dropped by 25 percent in the United States. Such an approach can be taken with bioavailable folate derivates. Accordingly, the invention contemplates a program of food fortification to achieve the prophylactic use of bioavailable folate derivates.

Existing food fortification with folic acid is intended to supply pregnant or periconceptional women with sufficient quantity of folate during the first trimester of pregnancy. This is because neural tube defects occur as a result of reduced folate levels during this point in fetus development. However, hydrocephalus is not so limited in time: i.e., reduced bioavailable folate metabolites may be responsible for causing hydrocephalus at any time during foetal development. Therefore bioavailable folate derivates can be used to prevent hydrocephalus when taken by women who are already pregnant. Hence a further embodiment of the invention is wherein the bioavailable folate derivative(s), or medicament comprising the bioavailable folate derivative(s), is supplied to a pregnant subject.

Such a beneficial effect can be seen even during quite late stages in pregnancy: up to the equivalent of 6 or 7 months or more in human pregnancy. Therefore, an embodiment of the aspects of the invention is wherein the bioavailable folate derivate(s) is supplied to a subject (possibly in the form of dietary supplements) during the second or third trimester of pregnancy. Such an administration regime would not have been anticipated until the present invention.

Where one or more bioavailable folate derivates is used to treat hydrocephalus, a subject identified as suffering from this disorder is administered a therapeutically effective quantity of the compound(s). In such a use the compound(s) is formulated and prepared as a therapeutic composition. The subject could be, for example, a developing fetus, neonate, infant, child, adolescent or adult. Where the subject is a developing fetus, then the bioavailable folate derivate(s) will be administered to the female pregnant with that fetus.

Hence the present invention encompasses both where the subject to be administered one or more bioavailable folate derivates has been diagnosed as suffering from hydrocephalus, as well as any subject who would benefit from the prophylactic administration of bioavailable folate derivates.

A further aspect of the invention provides a composition comprising two or more bioavailable folate derivative(s). Preferably the composition comprises folinic acid and tetrahydrofolate.

As mentioned above, the inventors consider that a combination of folinic acid and tetrahydrofolate (in the form of tetrahydrofolic acid) is surprisingly beneficial for the prevention or treatment of hydrocephalus as it is not toxic to cells and seems to reverse the development of hydrocephalus and improved cortex development. Preliminary data demonstrating this effect is briefly discussed in the accompanying examples. Therefore a combination of folinic acid and tetrahydrofolate has a surprising benefit not apparent until the present invention.

As mentioned below, optimal amounts of folinic acid and tetrahydrofolate present in the composition may be determined by those skilled in the art, and will vary with the strength of the preparation, the mode of administration, and the advancement of the disease condition to be treated with the composition.

As shown in the accompanying examples, 2.5 mg/kg of body weight of folinic acid leads to a reduction in likelihood of a fetus developing hydrocephalus. Therefore, a composition of this aspect of the invention may comprise around 2.5 mg/kg of body weight of each of the two or more bioavailable folate derivative(s). For a daily dose to be taken by a pregnant female of around 60 kg, then the composition may comprise around 150 mg of each of the two or more bioavailable folate derivative(s); for example 150 mg folinic acid and 150 mg tetrahydrofolate.

Compositions containing one or more bioavailable folate derivatives may take a number of different forms: for example, the composition may be in the form of a powder, tablet, capsule, liquid, ointment, cream, gel, hydrogel, aerosol, spray, micelle, liposome or any other suitable form that may be administered to a person or animal. It will be appreciated that the vehicle used to administer the compounds should be one which is well tolerated by the subject to whom it is given, and enables delivery of the compounds to the central nervous system.

The medicament may be used in a number of ways. For instance, systemic administration may be required in which case one or more bioavailable folate derivatives may be contained within a composition which may, for example, be ingested orally in the form of a tablet, capsule or liquid. Alternatively, the derivatives may be administered by injection into the blood stream. Injections may be intravenous (bolus or infusion) or subcutaneous (bolus or infusion). The compounds may also be administered by inhalation (e.g. intranasally).

It will be appreciated that the amount of bioavailable folate derivatives required is determined by biological activity and bioavailability which, in turn, depends on the mode of administration, the physicochemical properties of the compound employed and whether the compound is being used as a monotherapy or in a combined therapy. The frequency of administration will also be influenced by the above-mentioned factors and particularly the half-life of the compound within the subject being treated.

Optimal dosages to be administered may be determined by those skilled in the art, and will vary with the strength of the preparation, the mode of administration, and the advancement of the disease condition. Additional factors depending on the particular subject being treated will result in a need to adjust dosages, including subject age, weight, gender, diet, and time of administration.

Known procedures, such as those conventionally employed by the pharmaceutical industry (e.g. in vivo experimentation, clinical trials etc), may be used to establish specific formulations of compositions and precise therapeutic regimes (such as daily doses of the compounds and the frequency of administration).

Generally, a daily dose of between 0.01 μg/kg of body weight and 1.0 g/kg of body weight of bioavailable folate derivate may be used, more preferably, the daily dose is between 0.01 mg/kg of body weight and 100 mg/kg of body weight.

As shown in the accompanying examples, 2.5 mg/kg of body weight of folinic acid leads to a reduction in likelihood of a fetus developing hydrocephalus. Therefore, by way of example a suitable daily dose of folinic acid (an example of a bioavailable folate derivate) for treating hydrocephalus is between 1 mg/kg of body weight and 10 mg/kg of body weight, most likely 2.5 mg/kg of body weight.

Daily doses may be given as a single administration (e.g. a daily tablet for oral consumption or as a single daily injection). Alternatively the bioavailable folate derivative(s) may require administration twice or more times during a day. Purely as way of an example, folinic acid (an example of a bioavailable folate derivate) for treating hydrocephalus may be administered as two (or more depending upon the severity of the disorder) daily doses of between 0.5 mg/kg of body weight and 5 mg/kg of body weight in tablet form. A subject receiving treatment may take a first dose upon waking and then a second dose in the evening (if on a two dose regime) or at 3 or 4 hourly intervals thereafter. Alternatively a slow release device may be used to provide optimal doses to a subject without the need to administer repeated doses.

In the subject invention a “therapeutically effective amount” is any amount of one or more bioavailable folate derivates which, when administered to a subject, causes reduction, remission, or regression of the hydrocephalus. When used as a preventative, a “therapeutically effective amount” would be that which results in a reduced instance of hydrocephalus.

In the practice of this invention the “pharmaceutically acceptable vehicle” is any physiological vehicle known to those of ordinary skill in the art useful in formulating pharmaceutical compositions.

In one embodiment, the pharmaceutical vehicle may be a liquid and the pharmaceutical composition would be in the form of a solution. In another embodiment, the pharmaceutically acceptable vehicle is a solid and the composition is in the form of a powder or tablet. In a further embodiment, the pharmaceutical vehicle is a gel and the composition is in the form of a suppository or cream.

A solid vehicle can include one or more substances which may also act as flavoring agents, lubricants, solubilizers, suspending agents, fillers, glidants, compression aids, binders or tablet-disintegrating agents; it can also be an encapsulating material. In powders, the vehicle is a finely divided solid which is in admixture with the finely divided active ingredient. In tablets, the compound to be used, for example folinic acid, is mixed with a vehicle having the necessary compression properties in suitable proportions and compacted in the shape and size desired. The powders and tablets preferably contain up to 99% of the active ingredient. Suitable solid vehicles include, for example, calcium phosphate, magnesium stearate, talc, sugars, lactose, dextrin, starch, gelatin, cellulose, polyvinylpyrrolidine, low melting waxes and ion exchange resins.

Liquid vehicles are used in preparing solutions, suspensions, emulsions, syrups, elixirs and pressurized compositions. The bioavailable folate derivative(s) can be dissolved or suspended in a pharmaceutically acceptable liquid vehicle such as water, an organic solvent, a mixture of both or pharmaceutically acceptable oils or fats. The liquid vehicle can contain other suitable pharmaceutical additives such as solubilizers, emulsifiers, buffers, preservatives, sweeteners, flavoring agents, suspending agents, thickening agents, colors, viscosity regulators, stabilizers or osmo-regulators. Suitable examples of liquid vehicles for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the vehicle can also be an oily ester such as ethyl oleate and isopropyl myristate. Sterile liquid vehicles are useful in sterile liquid form compositions for parenteral administration. The liquid vehicle for pressurized compositions can be halogenated hydrocarbon or other pharmaceutically acceptable propellent.

Liquid pharmaceutical compositions which are sterile solutions or suspensions can be utilized by for example, intramuscular, epidural, intraperitoneal or subcutaneous injection. Sterile solutions can also be administered intravenously. Bioavailable folate derivative(s) may be prepared as a sterile solid composition which may be dissolved or suspended at the time of administration using sterile water, saline, or other appropriate sterile injectable medium. Vehicles are intended to include necessary and inert binders, suspending agents, lubricants, flavorants, sweeteners, preservatives, dyes, and coatings.

The bioavailable folate derivatives can be administered orally in the form of a sterile solution or suspension containing other solutes or suspending agents (for example, enough saline or glucose to make the solution isotonic), bile salts, acacia, gelatin, sorbitan monoleate, polysorbate 80 (oleate esters of sorbitol and its anhydrides copolymerized with ethylene oxide) and the like.

The bioavailable folate derivatives can also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

An embodiment of the present invention will now be described, by way of example, with reference to the accompanying drawings, in which:

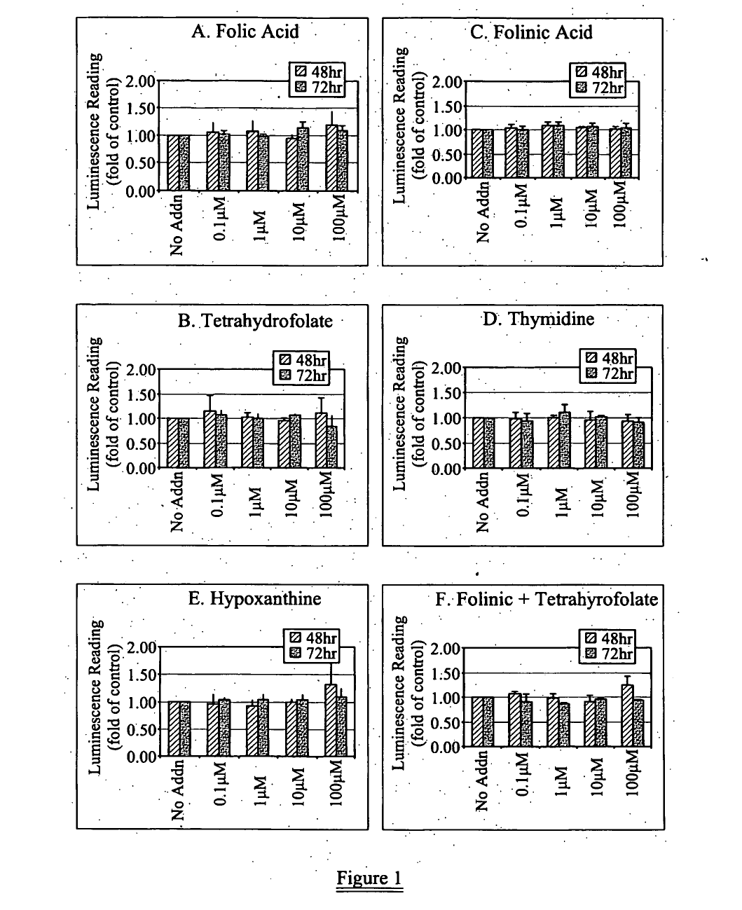

FIG. 1: Toxicity of Folate Derivatives In Vitro.

Cortical cells obtained from gestation age day 20 Wistar were plated in pre-treated 96 well plates at a starting density of 1×10⁵ cells/ml in supplemented neurobasal medium. After 24 hours the media was replaced with fresh media containing the concentrations of supplements as shown. The cultures were maintained at 37° C. in 5% CO₂. Proliferation of cells was measured after a further 24 or 48 hours using a luminescence-based assay. Luminescence results were normalised against control wells with no added supplements and presented as fold of control. Results shown are mean±SD of 1-2 experiments each performed in triplicate.

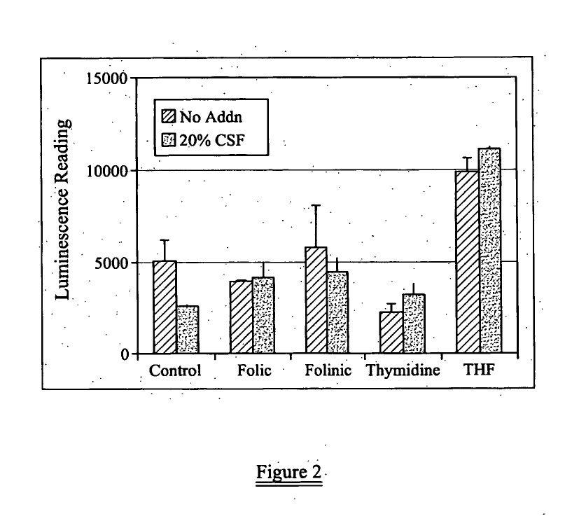

FIG. 2: Effect of Folate Derivatives on Hc Csf Mediated Inhibition of Proliferation.

Cortical cells obtained from gestation age day 20 Wistar were plated in pre-treated 96 well plates at a starting density of 1×10⁵ cells/ml in supplemented neurobasal medium. After 24 hours the media was replaced with fresh media containing the supplements as shown and/or 20% HC CSF. The cultures were maintained at 37° C. in 5% CO₂. Proliferation of cells was measured after a further 24 or 48 hours using a luminescence-based assay. Luminescence results were averaged and are shown are mean±SD of one preliminary experiment performed in triplicate.

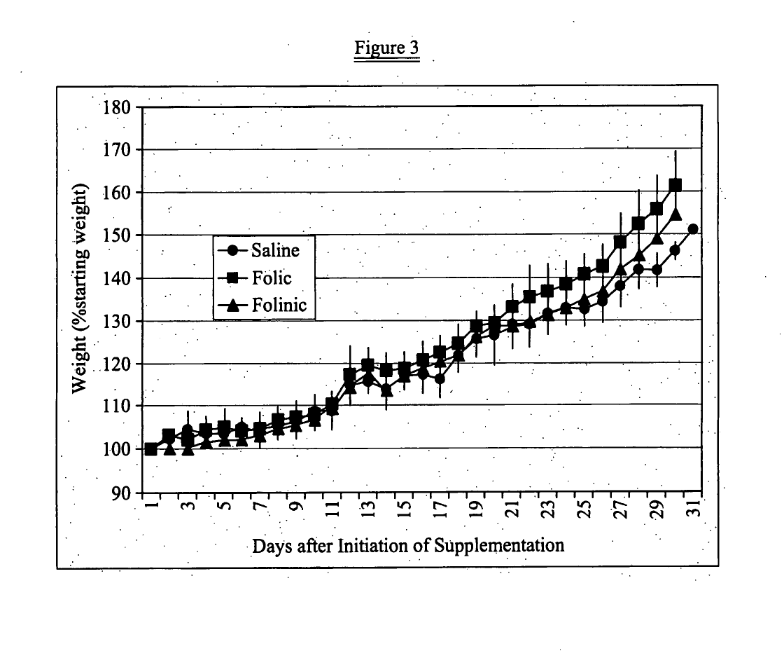

FIG. 3: Effect of Folic Acid and Folinic Acid Supplementation on Mother's Weight Through Pregnancy.

Mothers were injected daily with 2.5 mg/kg of the supplements folic acid or folinic acid or saline controls. Injections were started 14 days prior to mating and continued throughout pregnancy up to harvesting of the fetuses at day 20. Mothers were weighed at the same time each day and data converted to % of starting weight at day 0. Results were averaged across the 3 mothers (2 for saline treatment) and are plotted as shown mean±SD.

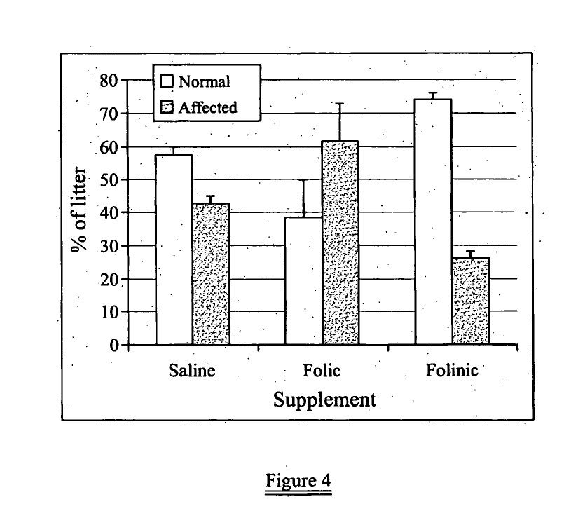

FIG. 4: Effect of Long Term Folic Acid and Folinic Acid Supplementation on the Numbers of Affected HC Fetuses.

Mothers were injected daily with 2.5 mg/kg of the supplements folic acid or folinic acid or saline controls. Injections were started 14 days prior to mating and continued throughout pregnancy up to harvesting of the fetuses at day 20. Fetuses were harvested on Day20 of gestation and each fetus was scored as affected HC or normal. The percentage of normal or affected fetuses in each litter was then calculated and results were averaged across the 3 mothers (2 for saline treatment) and are plotted as shown, mean±SD.

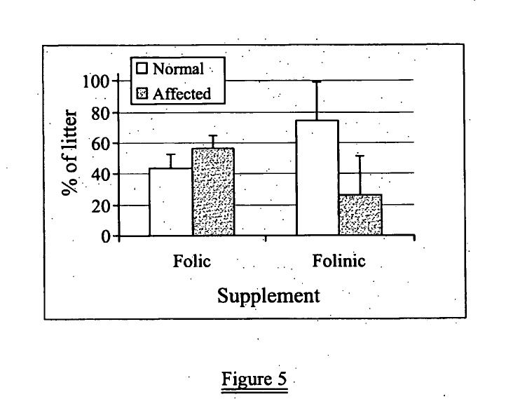

FIG. 5: Effect of Short Term Folic Acid and Folinic Acid Supplementation on the Numbers of Affected HC Fetuses.

Mothers were injected daily with 2.5 mg/kg of the supplements folic acid or folinic acid. Injections were started at E17 of the pregnancy and continued through the rest of the pregnancy up to harvesting of the fetuses at day 20. Fetuses were harvested on Day20 of gestation and each fetus was scored as affected HC or normal. The percentage of normal or affected fetuses in each litter was then calculated and results were averaged across the 3 mothers (2 for saline treatment) and are plotted as shown, mean±SD.

### EXAMPLE 1

### Bioavailable Forms of Folate: a Treatment to Restore Normal Development of the Brain in Hydrocephalus

Introduction

Hydrocephalus (HC) is a condition with multifactor aetiology. The incidence of HC is conservatively estimated at between 1/500 to 1/2000 live human births. The most devastating form of HC is that seen in fetuses and new born infants, early-onset HC, which has an incidence of around 1:1000. Treatment usually involves surgical drainage of CSF through insertion of ventriculoperitoneal shunts or ventriculostomy. Despite the benefits of shunting many of these patients have major neurological deficits which are linked to abnormal development of the cerebral cortex during fetal development rather than any damaging effects of fluid accumulation and raised intracranial pressure (although these will clearly be important in patients identified late and/or treated late).

The vast majority of studies on HC have concentrated on postnatal HC and the effects of fluid accumulation and raised intracranial pressure, restricted to primary mechanisms that include mechanical compression and stretching of brain parenchyma, ischemia and anoxia, cerebral oedema, and blood brain barrier dysfunction. There are also significant early consequences of fluid obstruction which cannot be attributed to pathological changes but result in abnormal development causing associated healthcare, educational and functional problems. Understanding the molecular mechanisms for these effects would lead to the prospect of therapeutic intervention to improve the developmental outcome even if patients still required fluid diversion.

Development of the cerebral cortex initially occurs from stem cells located in the germinal epithelium (GE) adjacent to the fluid filled ventricles of the brain. Our work has demonstrated that in early-onset HC, cerebrospinal fluid (CSF) obstruction occurring prenatally results in an arrest of cortical development through a direct effect on the cell cycle of stem cells in the germinal epithelium. We have demonstrated that:

1. Abnormal development of the cortex in the H-Tx rat occurs following (but not prior to) obstruction of CSF flow.

2. This is linked to a lack of proliferation of cortical GE cells resulting in reduced output of neurones into the cortical plate.

3. The lack of GE cell proliferation is a result of in vivo inhibition of the GE cells, as removing cells from the hydrocephalic brain and placing them into standard growth medium “releases them” from their in vivo arrested state and they show a normal ability to divide.

4. GE cells in HC fetal brains accumulate in the S-phase of the cell cycle compared to those from normal fetal brains.

5. Addition of CSF from HC fetuses to normal GE cells in vitro mimics the in vivo situation, causing inhibition of proliferation and S-phase blockade.

6. These in vitro effects of HC CSF are observed at CSF concentrations as low as 2.5% v/v.

Together these data point to CSF composition as one of the responsible elements in the developmental abnormalities associated with fetal-onset HC. The CSF is produced by the secretory epithelium of the choroid plexus. As well as fluid secretion, the choroid plexus secretes most of the proteins that are found within the CSF including many of the important growth factors that have been shown in vitro studies to be required for neurogenesis and brain development.

Models of Hydrocephalus

Currently there are no in vitro models of hydrocephalus. The H-Tx rat is a widely recognised model of early-onset HC and as such is very well characterised. Given the complexity of the processes of brain development and the number of different cell types involved, it is unlikely that any in vitro model could match all aspects of the process. Alternate animal models of HC involve experimentally induced HC by kaolin or cyanoacrylate injections into the cictema magna of rabbits, dogs, cats or rats. The H-Tx model is superior to these as it is a congenital model in which HC occurs (naturally) during early cortical neuronal development. Our colony is unique in the UK with other colonies being located in Switzerland, Germany, USA and Japan.

The features of the H-Tx model have been shown to be very similar to the commonest forms and presentations human fetal-onset HC, all of the other models listed above result in adult onset hydrocephalus (Table 1). Indeed, we have obtained limited samples of CSF from human infants shunted soon after birth and these samples have had similar effects of inhibiting proliferation, on rat cortical neurons growing in culture. These studies suggest that the components of CSF are similar in nature and effect between affected H-Tx rat and affected human.

Our research has brought us to a new understanding of EOHC. We have identified that folate metabolite levels are important in both the aetiology and outcome of hydrocephalus and that this may be applicable to at least 60% of all cases of EOHC in humans, the estimated number that do not resolve following third ventriculostomy, those in which aqueduct stenosis is clearly not the primary cause of the hydrocephalus.

Our results show that, with hydrocephalus, bio-availability of folate metabolites to cells of the developing cortex is reduced and that the synthesis pathways which make specific folate compounds are altered. Supplementation of the maternal diet with folinic acid could restore normal development of the cortex of the brain or even alleviate this debilitating condition completely.

Methods

Maintenance of H-Tx Colony

All experiments were conducted under license from the Home Office Animal Procedures Inspectorate. Both Wistar, Sprague-Dawey and H-Tx rat colonies were maintained on 12:12 light:dark cycle beginning at 8.00 am. They were kept at a constant temperature in large rat boxes with unrestricted access to food and water. The H-Tx colony was maintained through brother-sister mating between unaffected males and females whilst the Wistar and Sprague-Dawley colony were maintained through random pair matings. Timed mating was carried out by placing a male and female together in a box and checking for the presence of a vaginal plug every hour. The presence of a plug was taken to indicate successful mating and the time taken as gestational day zero.

Analysis Points

Timed pregnancies of H-Tx or Wistar rats were harvested from pregnant dams killed by intraperitoneal injection of sodium pentobarbitone. Fetuses were collected at gestation days 17-20 (LCMS experiments), day 19 (in vitro culture experiments) and day 20 (in vivo supplementation experiments), and decapitated. Brains were removed and processed as described below. Ventricular dilatation, opacity and clinical appearance of enlarged heads were used to identify affected H-Tx fetuses and pups.

Histological analysis confirmed that ventricular dilatation and aqueduct obstruction had occurred in our affected samples and was not present in our defined population of normal unaffected H-Tx.

Preparation of Cortical Cultures

Cortical hemispheres from the brains of embryonic day 20 Wistar fetuses were dissected in ice-cold sterile Phosphate Buffered Saline (PBS, pH 7.4). The tissue was digested in 0.25 Trypsin-EDTA (Sigma, UK) for 20 min at 37° C. Following incubation, Trypsin-EDTA was inactivated with Neurobasal medium (GIBCO) containing B27 medium Supplement (GIBCO). The suspension was subsequently centrifuged at 1700 rpm for 5 min and resuspended in fresh neurobasal medium. The tissue was then dissociated through sterile tips of decreasing bore (3 mm, 2 mm, 1 mm) followed by centrifuging at 1700 rpm for 5 min. The cells were resuspended in fresh medium and counted on a haemocytometer. The viability of the cells was greater than 95% when tested by trypan blue exclusion.

The dissociated cells were plated in pretreated 96 well plates at a density of 1×10⁵ cells ml-1 in Neurobasal medium which preferentially supports progenitor and neuronal cell types (not glial cells), containing B27 supplement, 2 mM glutamine and penicillin-streptomycin (0.1 mg ml-1). The cultures were maintained at 37° C. in 5% CO₂. After 24 hours recovery then the media was removed and supplements as shown and/or HC CSF was added to the wells for a further 24 or 48 hours. Proliferation of cells was then determined using the luminescence based Lumitech Vialight high sensitivity cell proliferation kit (as per manufacturers instructions). Measurements were made on a Multilabel Counter (Wallac Victor2 1420). Results are expressed as mean luminescence±SD (of at least triplicate samples).

Extraction of CSF

In experiments to determine the effect of CSF on normal, control cortical progenitor and neuronal cell cultures, CSF was removed from the cisterna magna of unaffected H-Tx and Wistar, or lateral ventricles of affected H-Tx fetuses at day E20. The samples were spun twice at 13000 rpm in a microcentrifuge for 15 min each time. The supernatant was removed, snap frozen in dry ice and stored at −80° C. This CSF was included in the assays at 20% v/v in normal culture medium/supplemented media. Results are expressed as mean luminescence±SD (of at least triplicate samples).

In Vivo Supplementation Protocols.

Female rats were handled for one week prior to the start of the supplementation protocol to decrease handling stress. At the end of the week, each female received daily subcutaneous injections of the folate supplements. Timed mating was performed at the end of the first week and supplementation was continued daily until gestational day E20. At E17, pregnant dams received an intraperitoneal injection of 2-bromodeoxyuridine (Sigma, UK) at 60 mg/kg body weight. Dams were killed at gestational day E20 and the fetuses recovered for analysis. Each fetus was uniquely identified to correlate CSF, blood and brain tissue analyses. Blood was also collected from each pregnant dam for future analysis of serum composition. Fetal brains were fixed in 4% paraformaldehyde in phosphate buffered saline for 12-24 hours before cryopreservation in sucrose (2 hours each in 10%, 20% and 30% sucrose solutions). Each was mounted on a chuck with CryoEmbed and rapidly frozen in isopentane cooled with dry ice. Sections were cut at 25 μm using a Leica DM300 cryostat, collected onto subbed slides and air dried overnight. BrdU labelled cells were identified using a monoclonal antibody as published previously (Mashayekhi et al). Additional sections were stained with methyl green and pyronine for histological analysis. Stained sections were photographed on a Lecia DMLB photomicroscope and analysed using Metaview software.

Results and Discussion.

As described above, previous data demonstrated that in the hydrocephalic fetus there was abnormal development of the cerebral cortex associated with a lack of proliferation and thus neuronal output from the germinal epithelium. This effect could be mimicked in vitro by addition of hydrocephalic CSF to normal germinal epithelium cells. The decreased proliferation was due to a blockage in the cell cycle whereby cells accumulated in the S phase.

Although there are many check points which regulate cell cycle kinetics, accumulation of cells in the S and G2/M phases of the cycle is unusual. This type of blockade is generally only observed in response to added drug treatments or DNA damaging agents. This led us to the hypothesis that the cycle blockade is a result of deficiencies in the metabolite pools within the cells, e.g. nucleotide pools such that DNA replication cannot proceed at the normal rate. One possibility was that the effects were linked to deficiencies in the availability of some folate derivatives (pteroylglutamic acid derivatives), leading to a lack of the nucleotide thymidine and other critical metabolites stemming from this critical family of metabolites.

Folate Uptake into Germinal Epithelium Cells is Inhibited by HC CSF.

Preliminary evidence that the inhibition of germinal matrix epithelium cells may be partly explained by a lack of bio-available folates in the CSF comes from in vitro studies looking at folate uptake into these cells. Data from preliminary experiments shows that there is a reduction in the amount of folate uptake into cells when HC CSF is added to the culture under the culture conditions where a reduction in proliferation and accumulation of cells in S-phase is observed. Uptake of [³H]-folic acid in the presence of 10% HC CSF is 53±13% (n=3) of that in the absence of added CSF.

One alternative explanation for the lack of folate uptake is that it could be a result either of lack of expression of a folate transporter protein at the cell surface, or inhibition of the activity of the transporters present. This is unlikely, as when HC germinal matrix cells are cultured under normal conditions in the absence of HC CSF, normal levels of proliferation are observed. This suggests that these cells do not have any intrinsic defects; the lack of activity is due to some external factors in their in vivo environment, namely the CSF.

Folate Metabolising Enzymes in the CSF

One possibility to account for a lack of bio-available folate in the HC CSF is that there is abnormal metabolism of these compounds in the HC CSF. Folate metabolism is a highly complex system and is involved in many critical aspects of both nucleotide and amino acid synthesis. We have now used proteomics-based approaches to identify protein differences between normal and affected CSF from a range of fetal ages over E17-E20 (the critical days for development of HC and manifestation of abnormal brain development). The LCMS data set was mined for any proteins with a role in folate metabolism. An exciting finding was that of the presence of formyltetrahydrofolate dehydrogenase (FMTHFDH) in affected CSF (Table 2).

In adult CSF folate levels are reported at 15-35 ng/ml and the majority of folate is in the form of 5-methyltetahydropteroylglutamic acid produced by the activity of a series of enzymes including 5,10-methylenetetrahydrofolate reductase and the vitamin B12 containing enzyme 5-methyltetrahyrdofolate homocysteine methyltransferase. Very little is known about the CSF in the developing brain but our data demonstrates that there is the potential for bio-available forms of folate to be synthesised in the CSF and delivered directly to the cells through the CSF. This means, during development, when the cell numbers are expanding rapidly, the CSF could provide metabolites to the cells to supplement intracellular production of critical compounds. In the absence or disruption of this extracellular system then cellular metabolism and thus proliferation would proceed at a much slower rate and abnormal cortical development would result.

These adverse effects may not just be manifest in the germinal epithelium. A lack of bio-available folate could result in reduced production of other critical cell types in the developing brain, for example, fluid output channels during development of the cortex. A reduction in the numbers or functionality of the generated fluid channels means that when the switch from embryonic to foetal fluid production occurs there is an imbalance between fluid production and outflow, resulting in fluid accumulation and hydrocephalus.

Together the above hypotheses concerning the consequences of abnormal folate metabolism in the hydrocephalic CSF could explain both the emergence of the condition itself and the subsequent abnormal brain development.

One of the most obvious differences between the HC CSF and normal CSF is the presence of FMTHFDH in CSF of hydrocephalic subjects. Increased activity of FMTHFDH would divert folate metabolites away from pyrimidine synthesis and lead to deficiencies in the nucleotide thymidine. This would provide a more specific explanation for the lack of proliferation observed in GE cells in the developing HC brain. However any imbalance in one part of a metabolic pathway will lead to disruption of the pathway as a whole, so the consequences of the activity of FMTHFDH may be manifest in some of the other interlinking pathways and lead, for example, to a diversion of bio-available folates from pyrimidine synthesis into branches of the system involved in protein synthesis.

On the basis of the above data, we formulated the hypothesis that supplementation of the mother with bio-available forms of folate may alleviate this condition. Specifically:—Supplying a mother with folinic acid (which would bypass any enhanced activity of FTHFDH and allow pyrimidine synthesis to occur) or other bioavailable forms of folate (see list below) would allow restoration of the balance of folate metabolism in the developing brain leading to:—

1. Prevention of hydrocephalus by allowing normal development of output channels;

2. Allow normal brain development by-passing the enzymatic blockade of normal folate metabolism; and,

3. Treat hydrocephalus by rescuing lost development following late diagnosis of hydrocephalus.

Preliminary experiments have been undertaken to test this hypothesis.

In Vitro Testing of Supplements.

a) Toxicity Testing.

In vitro cortical cultures were incubated with a range of supplements including folic acid, folinic acid, tetrahydrofolate, hypoxanthine, thymidine and combinations of folinic acid with tetrahydrofolate. The concentrations of the supplements ranged from 100 nM through to 100 μM. This data is presented in FIG. 1.

Allowing for the variability in this preliminary dataset, there is no significant evidence of any toxicity to the culture. It is also worth noting that the effects observed are compared to control cells at the same time points. These cells have proliferated over the 48/72 hour period and there are thus many more cells in the wells compared to the original starting concentration of 10⁴/well. Thus any minimal effects are associated with a reduction in proliferation of the cells, not significant cell death.

This data suggests that these supplements are not toxic to the cortical cells.

b) Effect of Supplements on Inhibition of Cellular Proliferation by HC CSF

Our previous data has demonstrated the inhibitory qualities of HC CSF. This can also be clearly seen in FIG. 2. In all cases where supplements were added along with the HC CSF then no CSF mediated inhibition of proliferation was observed. Data is variable with respect to the extent of proliferation under all of the supplements tested, but there is no evidence of any inhibition by HC CSF of supplemented wells compared to control wells.

Together, this data points to the fact that the supplements are non toxic and do have the ability to reverse HC CSF mediated inhibition of cortical cell function and thus potentially to restore normal levels of bio-available folate in the developing brain.

In Vivo Testing of Supplements.

The hypothesis was tested further using the H-Tx model of EOHC. Mothers were injected daily with 2.5 mg/kg of the supplements folic acid (3 mothers-31 pups) or folinic acid (3 mothers-35 pups) or saline controls (2 mothers-20 pups). Injections were started 14 days prior to mating and continued throughout pregnancy up to harvesting of the fetuses at day 20. The weight of the mothers was monitored daily and there were no significant differences in the weight of the mothers across the supplement protocols, although the folic acid and folinic acid supplemented mothers appeared to have slightly increased weight compared to saline treated animals (FIG. 3).

Each fetus was scored as affected HC or normal and brains were processed for future analysis. Data from a first experiment is shown in FIG. 4 with the percentage of normal or affected fetuses in the litter recorded. The rate of HC in the saline controls is 45%. In comparison, folic acid supplementation increases this to 60% but this increase is not significant in this one experiment. In contrast there is a significant reduction (p<0.05) in the incidence of HC in the folinic acid supplemented animals to 30% affected with HC.

One issue with human dietary supplements is that mothers are often unaware of the pregnancy in the early stages and do not always plan or adhere to pre-pregnancy dietary regimes. Given that the condition of HC is not manifest until the later stages of gestation, then we expect that it may be possible to alleviate (or prevent) the condition (or its effects on brain development) even if supplementation were started later on in the pregnancy. Preliminary experiments to test this were carried out on H-Tx rats starting at E17 of gestation the age at which blockage of CSF flow is initially manifest. Mothers were supplemented (by injection) with folic or folinic acid as before and results are shown in FIG. 5. It appears that folinic acid is once again capable of reducing the incidence of HC in the supplemented mothers, suggesting that supplementation post-conception could also have beneficial effects.

Combination Treatments

We then assessed whether combinations of bioavailable folate derivatives can have a beneficial effect on hydrocephalus and cortex development.

Preliminary data from two litters of H-Tx model of EOHC supplied with a combination of folinic acid and tetrahydrofolic acid shows that 13% are affected with hydrocephalus, while 83% are unaffected and the remaining could not be classified on gross morphology.

### CONCLUSIONS

Taken together the above data provides evidence that the hypothesis that maternal supplementation with the bio-available derivatives of folate (and combinations of such compounds) will have beneficial effects in terms of a reduction in the likelihood of HC developing in a fetus is correct. The doses used in the in vivo study are 2.5 mg/kg. Given an average weight for a rat of around 330 g and a rat blood volume of around 10 ml, this equates to an absolute maximum circulating concentration of approx 0.2 mg/ml (or approx 500 uM). Obviously concentrations achieved in the CSF of the developing fetus will be much lower than this. It is possible that the use of higher concentrations of supplements would lead to 100% normal fetuses.

Pre-pregnancy folic acid supplementation of female diets has been well established as a mechanism for reducing the incidence of neural tube defects, namely spina bifida, however there is no evidence that the incidence of HC is affected in this population. In fact, data from the few reports where HC was explicitly analysed confirms that supplementation with folic acid does not have any significant effect on the incidence of HC. Our in vivo data from the rat actually points to a potentially negative effect of folic acid on the HC outcome. However it must be borne in mind that the H-Tx rat model has a genetic susceptibility to develop HC. Thus in this susceptible background negative effects of folic acid supplementation are apparent. In the general population folic acid does not lead to increased incidences of HC. We hypothesise that the reason why folic acid does not have an effect, whereas other bio-available forms of this metabolite do, is linked to the balance of metabolism of folates within the CSF. Folic acid supplementation is insufficient if the enzyme imbalance in folate metabolism within the HC CSF is such that this folic acid cannot be converted to the required bio-available forms. Alternatively, the transfer of other derivatives into fetal CSF may be much more efficient compared to folic acid. Indeed competition at the level of transport could explain why folic acid has a negative effect in the H-Tx model.

TABLE 1
(data from multiple sources): Comparison of the Characteristics of Early
Onset Hydrocephalus in Humans with Those of H-Tx Rats.

| Characteristic | Affected H-Tx rat | Human early-onset HC |
|---|---|---|
| CSF blockage occurs prior to | 100% | 100% |
| birth | | |
| Blockage occurs before or during | 100% | Not determined but is observed |
| cortical development | | on 16 week scans which is post |
| | | the initial development of the |
| | | cortex. |
| Blockage of CSF flow by | 100% | Majority |
| stenosis of the cerebral aqueduct | | |
| Reduced thickness of final fully | 100% | 100% |
| developed cortex | | |
| Reduced number of cortical | 100% | Not determined but suspected in |
| layers | | all cases. |
| Deficient cortical development | 100% | Not determined but assumed due |
| prior to raised pressure | | to flexible fetal skull |
| Neurological deficits persists | 100% | 100% |
| despite shunting | | |
| Cortical architectural changes not | Cortical thickness shows some | Specifics not determined but |
| reversed by shunting | recovery due to gliogenesis but | neurological deficits persist. |
| | not neurogenesis | |

TABLE 2
Enzymes that metabolise folate in E20 fetal CSF (LCMS data)

| Enzymes Detected | Normal | HC |
|---|---|---|
| METHYLENETETRAHYDROFOLATE | Yes | Yes |
| DEHYDROGENASE | | |
| 5-METHYLTETRAHYDROFOLATE- | Yes | Yes |
| HOMOCYSTEINE S-METHYLTRANSFERASE | | |
| 10-FORMYLTETRAHYDROFOLATE | No | Yes |
| DEHYDROGENASE (FMTHFDH) | | |

1. One or more bioavailable folate derivatives, or salts thereof, for the prevention or treatment of hydrocephalus.

2. The bioavailable folate derivatives, or salts thereof, of claim 1 wherein the bioavailable folate derivative(s) is or is any combination of: folinic acid, tetrahydrofolate, thymidine, 10-formyltetrahydrofolate or methyltetrahydrofolate, or salts thereof.

3. The bioavailable folate derivatives, or salts thereof, of claim 2 wherein the bioavailable folate derivative is folinic acid, or a salt thereof.

4. The bioavailable folate derivatives, or salts thereof, of claim 3 wherein the salt is calcium folinate or sodium folinate.

5. The bioavailable folate derivatives, or salts thereof, of claim 1 wherein the bioavailable folate derivative(s) comprises a combination of folinic acid and tetrahydrofolate.

6. The bioavailable folate derivatives, or salts thereof, of claim 1 wherein the bioavailable folate derivative(s) is formulated as a dietary supplement.

7. The bioavailable folate derivatives, or salts thereof, of claim 1 wherein the hydrocephalus is Early Onset hydrocephalus.

8. The bioavailable folate derivatives, or salts thereof, of claim 1 wherein the bioavailable folate derivative(s) is supplied to a human subject.

9. The bioavailable folate derivatives, or salts thereof, of claim 8 wherein the human subject is a pregnant female.

10. The bioavailable folate derivatives, or salts thereof, of claim 8 wherein the human subject is a developing fetus.

11. The bioavailable folate derivatives, or salts thereof, of claim 10 wherein the bioavailable folate derivative(s) is supplied to a female pregnant with that fetus.

12. The bioavailable folate derivatives, or salts thereof, of claim 9 or 11 wherein the bioavailable folate derivative(s) is supplied to the pregnant female during the second or third trimester of pregnancy.

13. The bioavailable folate derivatives, or salts thereof, of claim 8 wherein the human subject is a neonate, an infant, a child, an adolescent or an adult.

14. A composition comprising two or more bioavailable folate derivative(s).

15. The composition of claim 14 wherein the composition comprises folinic acid and tetrahydrofolate.

16. A method of preventing or treating hydrocephalus comprising administering one or more bioavailable folate derivatives, or salts thereof, to a subject in need of said treatment.
